(19) United States
(12) Patent Application Publication
(10) Pub. No.: US 2026/0265831 A1
(43) Pub. Date: Sep. 10, 2026

(54) USE OF T CELL TOLERANT FRACTION AS A PREDICTOR OF IMMUNE-RELATED ADVERSE EVENTS

(71) Applicant: THE BOARD OF REGENTS OF THE UNIVERSITY OF TEXAS SYSTEM, Austin, TX (US)

(72) Inventors: Jared OSTMEYER, Austin, TX (US); David GERBER, Austin, TX (US)

(73) Assignee: THE BOARD OF REGENTS OF THE UNIVERSITY OF TEXAS SYSTEM, Austin, TX (US)

(21) Appl. No.: 19/127,072

(22) PCT Filed: Nov. 2, 2023
(86) PCT No.: PCT/US2023/078520

§ 371 Date: May 2, 2025

Related U.S. Application Data
(60) Provisional application No. 63/382,257, filed on Nov. 3, 2022.

(51) Int. Cl. C12Q 1/6886; A61K 39/00; A61P 35/00; C07K 16/28

(52) U.S. Cl. CPC C12Q 1/6886; A61P 35/00; C07K 16/2818; C07K 16/2827; A61K 2039/505; A61K 2039/507; C07K 2317/21; C07K 2317/24; C12Q 2600/118; C12Q 2600/156; C12Q 2600/158

(57) ABSTRACT

Provided herein are methods of predicting a risk of developing an immune-related adverse event (irAE) from an immune checkpoint inhibitor (ICI) therapy. The methods include the classification of T cell receptor p genes as productive TCRβ gene or repaired TCRβ gene and calculating a tolerant fraction (TF) score. The classification of the TCRβ genes indicates the relative presence of non-tolerant T cells that are likely to recognized self-antigen after treatment with an ICI therapy. The relative presence of on-tolerant T cell is indicative of the risk of developing irAE.

### CROSS-REFERENCE TO RELATED APPLICATIONS

This application claims benefit of priority under 35 U.S.C. § 119(e) of U.S. Provisional Application No. 63/382,257, filed Nov. 3, 2022. The disclosure of this application is considered part of and is herein incorporated by reference in the disclosure of this application in its entirety.

### STATEMENT REGARDING GOVERNMENT FUNDING

This invention was made with government support under Grant Nos. 1U01AI156189-01, awarded by the National Institute of Allergy and Infectious Disease. The government has certain rights in the invention.

### BACKGROUND OF THE DISCLOSURE

### Field of the Disclosure

The present invention relates generally to immune checkpoint inhibitors (ICIs), and more specifically to ICIs-related toxicity.

### Background Information

ICIs have emerged as promising treatments for many cancer types. However, these therapies can elicit unpredictable and potentially severe autoimmune toxicities termed immune-related adverse events (irAE). As ICI regimens move from advanced disease to early-stage, curable settings, concerns over irAE—which in rare cases may be permanent or fatal—have increased. Furthermore, the growing use of combination ICI regimens, which are now approved for melanoma, lung cancer, and mesothelioma, raises concerns for increased ICI incidence and severity. Complicating these considerations, optimal monitoring for irAE remains unknown. Additionally, diagnosis of irAE may be more challenging than diagnosing toxicities of conventional chemotherapy or molecularly targeted therapies.

Approved ICI, including those targeting cytotoxic T lymphocyte antigen 4 (CTLA4), programmed death 1 (PD1) and PD1 ligand (PDL1), and lymphocyte antigen 3 (LAG3), mediate their effects through T cells. Accordingly, T cell characterization—in particular T cell receptor (TCR) sequencing—has been studied as a means to predict risk of irAE. TCRβ clonality and diversity can predict an irAE.

Immune tolerance refers to the immune system's unresponsiveness to substances that would otherwise elicit an immune response. This state arises from prior exposure to an antigen and may be induced centrally (in the thymus or bone marrow) or peripherally (in lymph nodes or other tissues). Immune tolerance represents a key tenet of normal physiology, as it allows the immune system to distinguish self from non-self. Conversely, deficits in tolerance may result in autoimmune disease. Because irAE represent ICI-associated autoimmunity, the present disclosure determined whether T cell tolerance—characterized according to productive or non-productive TCRβ genes—was associated with these toxicities.

### SUMMARY OF THE DISCLOSURE

Provided herein is a tolerant fraction score, methods of calculating it, and methods of use thereof to predict the risk of developing autoimmune toxicity from immune checkpoint inhibitor therapies.

An embodiment provides a method of predicting a risk of developing an immune-related adverse event (irAE) from an immune checkpoint inhibitor (ICI) therapy in a subject.

The method can comprise a) classifying T cell receptor p (TCRβ) genes of a cell from the subject as productive TCRβ gene or repaired TCRβ gene; b) classifying TCRβ genes of a cell from a pool of donors as a productive TCRβ gene or repaired TCRβ gene; and c) calculating a tolerant fraction (TF) score, wherein the TF score is a ratio of a productive fraction (F_{PROD}) and a total fraction (F_{TOTAL}), wherein F_{PROD} the number of TCRβ genes identified as productive TCRβ genes from the subject that overlap with TCRβ genes identified as productive TCRβ genes from the pool of donors F_{TOTAL} is sum of (i) the number of TCRβ genes identified as productive TCRβ genes from the subject that overlap with TCRβ genes identified as productive TCRβ genes from the pool of donors and (ii) the number of TCRβ genes identified as productive TCRβ genes from the subject that overlap with TCRβ genes identified as repaired TCRβ genes from the pool of donors, thereby predicting a risk of developing irAE from ICI therapy.

Productive TCRβ genes can produce a T cell receptor (TCR) that is tolerant to self-antigens. Repaired TCRβ genes can produce a TCR that is not tolerant to self-antigens. TCRs that are not tolerant to self-antigens can induce auto-immune toxicity. The method can further comprise comparing the TF score to a TF threshold, wherein a TF score greater than the TF threshold indicates a lower risk of developing irAE from ICI therapy and a TF score lower than the TF threshold indicates a higher risk of developing irAE from ICI therapy. A TF threshold can be about 82.5, or about 70, 75, 80, 81, 82, 82.5, 83, 85, or 90%. The risk can be predicted prior to a treatment with an ICI therapy. The method can further comprise administering to the subject having a lower risk of developing irAE an ICI therapy.

Classifying TCRβ genes can comprise: a) obtaining TCRβ genes sequences comprising multiple gene segments and somatic alterations; b) translating at least one of the multiple gene segments or somatic alterations into an amino acid sequence; c) identifying a TCRβ gene encoding an amino acid sequence capable of antigen recognition as a productive TCRβ gene, d) identifying a TCRβ gene without an amino acid sequence capable of antigen recognition as a non-productive TCRβ gene, e) repairing the amino acid sequence of a TCRβ gene identified as non-productive to generate a repaired TCRβ gene capable of antigen recognition, and f) classifying the TCRβ gene as a productive TCRβ genes or as a repaired TCRβ genes. The gene segments can be selected from the group consisting of variable (V) gene segments, diversity (D) gene segments, joining (J) gene segments, and any combination thereof. The non-productive TCRβ gene can be a TCRβ gene with out-of-frame gene segments or a TCRβ gene with a stop codon in a somatic junction between gene segments. Repairing non-productive TCRβ gene can comprise adding or removing one or more nucleotides at a somatic junction between gene segments to bring the gene segments in a same reading frame and/or mutating a nucleotide in a somatic region between gene segments to convert a stop codon into an amino acid. The TCRβ gene sequence can comprise a complimentary determining region 1 (CDR1) sequence of the TCRβ gene, a CDR2 sequence of the TCRβ gene, a CDR3 sequence of the TCRβ gene, a combination thereof, or a sequence of a complete TCRβ gene. The TCRβ gene sequence can comprise a CDR3 sequence of the TCRβ gene. The method can further comprise removing the first three amino acids and the last three amino acids of the CDR3 sequences from the TCRβ gene sequence. Obtaining a TCRβ gene sequence can comprise sequencing TCRβ genes from a peripheral blood mononucleated cell sample from the subject. Obtaining a TCRβ gene sequence can further comprise isolating T cells from the sample. Isolating T cells can be by cell sorting and/or RNA expression. T cells can be non-regulatory T cells.

The cell can be a peripheral blood mononucleated cell from a subject having cancer. The cancer can be selected from the group consisting of melanoma, prostate cancer, non-small cell lung cancer, mesothelioma, bladder cancer and renal cancer. The ICI therapy can be selected from the group consisting of ipilimumab, tremelimumab, atezolizumab, avelumab, durvalumab, nivolumab, pembrolizumab, ipilimumab plus nivolumab, and combinations thereof.

### BRIEF DESCRIPTION OF THE DRAWINGS

The accompanying drawings are included to provide a further understanding of the methods and compositions of the disclosure, are incorporated in, and constitute a part of this specification. The drawings illustrate one or more embodiments of the disclosure, and together with the description serve to explain the concepts and operation of the disclosure.

FIGS. 1A-1B illustrate the hypothesis and the methods of the study. FIG. 1A is a schematic representation of the hypothesis of this study. Patients with a higher percentage of tolerant T cells are anticipated to be at a lower risk of developing an irAE from ICI therapy because their T cells will not recognize self-antigens even after ICI therapy. Conversely, patients with a lower percentage of tolerant T cells are anticipated to be at a higher risk of developing an irAE from ICI therapy. FIG. 1B is a schematic representation of the method of this study. Non-productive and productive TCRβ genes are separated from 786 subjects with no known disease. The pool of non-productive TCRβ genes cannot express and therefore can be non-tolerant. The pool of productive TCRβ genes can express and therefore are tolerant. We characterize each cancer patient according to the relative overlap of their productive TCRβ genes with that of the tolerant pool (from the 786 subjects), which we call the tolerant fraction.

FIGS. 2A-2C illustrate TCRβ gene sequences reveal germline encoded V, D, and J gene segments as well as somatic alterations that occur during V(D)J recombination. FIG. 2A shows productive TCRβ genes found in peripheral blood can be translated to an amino acid sequence. FIG. 2B shows TCRβ genes found in peripheral blood with out-of-frame V and J gene segments do not express a functioning receptor for T cell selection. This example of a non-productive TCRβ gene can be computationally repaired by deleting somatic nucleotides. FIG. 2C shows TCRβ genes found in peripheral blood encoding a stop codon in a somatic junction also do not express a functioning receptor for T cell selection. This example of a non-productive TCRβ gene can be computationally repaired by modifying somatic nucleotides.

FIGS. 3A-3F illustrate the steps for processing the CDR3 of each TCRβ. FIG. 3A shows the TCR consists of a TCRα and TCRβ chain that each contribute a CDR3 for antigen recognition. Because only the TCRβ chain is sequenced, we discard all TCRβ chains with a “short” CDR3 based on the hypothesis that TCRβ chains with a short CDR3 cannot contribute to antigen recognition. FIG. 3B shows first and last three amino acid residues are trimmed from each CDR3 based on previously published observations from 3D X-ray crystallographic structures that these residues do not make direct contact with antigen and therefore are not anticipated to contribute to antigen recognition. FIG. 3C shows overlap between two sets of TCRβ sequences is determined by placing TCRβ sequences with identical trimmed CDR3 sequences into the overlapping region. FIG. 3D shows a plot of CDR3 from paired chains of individual cells reveal no correlation between CDR3 lengths (Pearson correlation coefficient r=0.0062≈0.0). Cells with more than one of either chain were excluded from analyses. FIG. 3E shows a Rendering of the 3-dimensional X-ray crystallographic structure with PDB ID 4jrx shows a specific TCR in contact with antigen, with the longer chain achieving more contact with antigen. The CDR3 of the TRA chain (blue) is 14 residues with 8 antigen (red) contact residues (57%) (defined as being within 5 Å of the antigen), whereas the CDR3 of the TRB chain (green) is 11 residues with 3 antigen (red) contact residues (27%). FIG. 3F shows AUROC (area under the receiver operating characteristic) curve measuring the ability of the TRB tolerant fraction to predict irAEs shown for different length cutoffs for discarding short CDR3 sequences. The performance is best when short CDR3 sequences are discarded.

FIGS. 4A-4C illustrate that the tolerant fraction is a predictor of an irAE. FIG. 4A shows the tolerant fraction for each patient with a grade ≥2 irAE (red square) versus a grade 0-1 irAE (blue triangle) shown for different ICI therapies. The threshold (dashed line) is picked to maximize the average of the sensitivity and specificity, achieving a sensitivity of 18/24=75% and a specificity of 39/53≈73.6%. FIG. 4B is a plot of the ROC. The area under the curve (AUC) is 0.79. FIG. 4C shows T cells enriched for napsin A, an antigen that potentially contributes to irAE in lung cancer patients, show a reduced tolerant fraction score. This is consistent with expectations that napsin A-specific T cells are not tolerant.

FIGS. 5A-5D illustrate TCRβ diversity and clonality are weak predictors of an irAE. FIG. 5A shows the TCRβ diversity for each patient with a grade ≥2 irAE (red square) versus grade 0-1 irAE (blue triangle) shown for different ICI therapies. The threshold (dashed line) is picked to maximize the average of the sensitivity and specificity, achieving a sensitivity of 43.4% and a specificity of 83.3%. FIG. 5B is a plot of the ROC. The area under the curve (AUC) is 0.60. FIG. 5C shows the TCRβ clonality for each patient with a grade ≥2 irAE (red square) versus grade 0-1 (blue triangle) shown for different ICI therapies. The threshold (dashed line) is picked to maximize the average of the sensitivity and specificity, achieving a sensitivity of 75.0% and a specificity of 58.5%. FIG. 5D is plot of the ROC. The area under the curve (AUC) is 0.62.

FIGS. 6A-C illustrate that the tolerant fraction is a predictor of an irAE. FIG. 6A The 0%, 25%, 50%, 75%, and 100% quartiles shown using box-and-whisker plots of the tolerant fraction for patients with grade ≥2 irAEs (red) versus grade 0-1 irAEs (blue). The dot represents an outlier (exceeding 1.5 the interquartile range). FIG. 6B ROC plot demonstrating the true and false positive rates resulting from different thresholds of the tolerant fraction for distinguishing the two outcomes (grade ≥2 versus grade 0-1 irAEs). The area under the curve (AUC) is 0.79. FIG. 6C T cells enriched for napsin A, an antigen that potentially contributes to irAEs in lung cancer patients, show a reduced tolerant fraction score. This is consistent with expectations that napsin A-specific T cells are not tolerant.

FIG. 7 illustrates that the tolerant fraction as a predictor of an irAE for different types of ICI. The 0%, 25%, 50%, 75%, and 100% quartiles are shown using box-and-whisker plots of the tolerant fraction for patients with grade ≥2 irAEs (red) versus grade 0-1 irAEs (blue). Dots represent outliers (exceeding 1.5 the interquartile range). In the combination therapy group, only three patients experienced grade 0-1 irAE, hence the lack of error bars in the plot for this category.

FIGS. 8A-B illustrate the TRB tolerant fraction calculated for various T cell populations. FIG. 8A TRB tolerant fraction for peripheral blood (blue circles) and thymus (red squares) in an immunologically healthy population. Samples were collected from pediatric patients undergoing corrective surgery for congenital cardiac defects. Peripheral blood and thymus samples were available for patients 1 to 4, while only thymus samples were available for patients 5 to 8. FIG. 8B Disease-specific TRB from patients with infection (influenza, coronavirus) (blue circles) and autoimmune disease (type 1 diabetes mellitus [T1DM], and multiple sclerosis [MS]) (red squares). TRBs represent aggregates from multiple patients/experiments within a disease group.

FIGS. 9A-D illustrate TRB diversity and clonality as predictors of irAE. FIG. 9A The 0%, 25%, 50%, 75%, and 100% quartiles shown using box-and-whisker plots of TRB diversity for patients with grade ≥2 irAEs (red) versus grade 0-1 irAEs (blue). Dots represent outliers (exceeding 1.5 the interquartile range). See methods section for how to interpret the p-value. FIG. 9B ROC plot demonstrating the true and false positive rates resulting from different thresholds of TRB diversity for distinguishing the two outcomes (grade ≥2 versus grade 0-1 irAE). The area under the curve (AUC) is 0.60. FIG. 9C The 0%, 25%, 50%, 75%, and 100% quartiles shown using box-and-whisker plots of TRB clonality for patients with grade ≥2 irAEs (red) versus grade 0-1 irAEs (blue). Dots represent outliers (exceeding 1.5 the interquartile range). See methods section for how to interpret the P-value. FIG. 9D ROC plot demonstrating the true and false positive rates resulting from different thresholds of TRB clonality for distinguishing the two outcomes (grade ≥2 versus grade 0-1 irAEs). The area under the curve (AUC) is 0.62.

FIG. 10 is an illustration of T cell selection showing why productive TCRβ genes are assumed to be tolerant, while non-productive TCRβ genes are not. Productive (P) TCRβ genes express a receptor (denoted as protrusions from each cell). The receptor determines which developing T cells are removed during T cell selection (e.g., red and blue are removed; purple is not). Therefore, productive TCRβ genes must express a receptor chain that is tolerant (e.g., purple). The non-productive (NP) TCRβ genes do not express a receptor. The unexpressed TCRβ genes do not determine which T cells are removed during T cell selection. Consequently, the non-productive TCRβ genes do not have to be tolerant. Both productive and non-productive TCRβ genes are simultaneously captured during TCRβ gene sequencing.

FIG. 11 is a schematic summarizing the steps for calculating the Tolerant Fraction.

### DETAILED DESCRIPTION

The present disclosure provides methods of predicting immune-related adverse events (irAEs) resulting from immune checkpoint inhibitor therapies. The methods include calculating a tolerant fraction (TF) score.

### Overview

Immune checkpoint inhibitor (ICI) therapies are among the most promising cancer therapies but may cause unpredictable and potentially severe autoimmune toxicities termed immune-related adverse events (irAE). Because T cells mediate the effects of ICI, T cell profiling may provide insight into irAE risk. Here a new metric was evaluated—the T-cell tolerant fraction—as a predictor of future irAE.

Patients intended for ICI therapy were enrolled in a prospective study to identify biomarkers predictive of future irAE. In this study the T-cell receptor beta locus (TRB; previously named T cell receptor β-chain) was examined. Pretreatment blood samples were obtained and subjected to TRB sequencing. Each patient is characterized by calculating the T cell tolerant fraction, as defined in the manuscript, from the patient's TRB sequences. The tolerant fraction is then assessed as a predictor of future irAE. The tolerant fraction is compared to TRB clonality and diversity that previous studies determined are predictors of irAE.

T cell receptor (TCR) p gene sequencing from baseline pre-treatment blood samples was performed by Adaptive Biotechnologies. Productive TCRβ genes that were in-frame and not containing a stop codon were considered to be tolerant, while non-productive TCRβ genes (either out-of-frame or containing a stop codon in the rearrangement) were not considered tolerant. The tolerant fraction was calculated by dividing the number of tolerant TCRβ by the total number of measured TCRβ. irAE were characterized by Common Terminology Criteria for Adverse Events and categorized as Grade 0-1 (not clinically significant) or Grade ≥2 (clinically significant). A one-sided Mann-Whitney U test was used to calculate P values and adjusted for repeated testing using Bonferroni correction.

A total of 77 cases from our prospective institutional cohort and three published studies were included, of which 43 (56%) received anti-CTLA4 therapy, 19 (25%) received anti-PD1/PDL1 therapy, and 15 (19%) received combined anti-CTLA4+anti-PD1/PDL1. The tolerant fraction was significantly lower in cases with clinically significant irAE (P<0.001). Using a tolerant fraction cut-off of 85.2%, sensitivity was 75%, specificity was 74%, and area under the receiver operating curve (AUC) was 0.79. In the same cohort, T cell clonality had an AUC of 0.62, and T cell diversity had an AUC of 0.60.

Among patients receiving diverse types of ICI, baseline T-cell tolerant fraction predicts future irAE and achieves better results than T cell clonality or diversity.

Methods of Predicting a Risk of Developing irAEs from ICI Therapies

Provided herein are methods of predicting a risk of developing an immune-related adverse event (irAE) from an immune checkpoint inhibitor (ICI) therapy in a subject.

The method can comprise a) classifying T cell receptor p (TCRβ) genes of a cell from the subject as productive TCRβ gene or repaired TCRβ gene; b) classifying TCRβ genes of a cell from a pool of donors as a productive TCRβ gene or repaired TCRβ gene; and c) calculating a tolerant fraction (TF) score, wherein the TF score is a ratio of a productive fraction (F_{PROD}) and a total fraction (F_{TOTAL}), wherein F_{PROD} the number of TCRβ genes identified as productive TCRβ genes from the subject that overlap with TCRβ genes identified as productive TCRβ genes from the pool of donors F_{TOTAL} is sum of (i) the number of TCRβ genes identified as productive TCRβ genes from the subject that overlap with TCRβ genes identified as productive TCRβ genes from the pool of donors and (ii) the number of TCRβ genes identified as productive TCRβ genes from the subject that overlap with TCRβ genes identified as repaired TCRβ genes from the pool of donors, thereby predicting a risk of developing irAE from ICI therapy.

Productive TCRβ genes can produce a T cell receptor (TCR) that is tolerant to self-antigens. Repaired TCRβ genes can produce a TCR that is not tolerant to self-antigens. TCRs that are not tolerant to self-antigens can induce auto-immune toxicity. The method can further comprise comparing the TF score to a TF threshold, wherein a TF score greater than the TF threshold indicates a lower risk of developing irAE from ICI therapy and a TF score lower than the TF threshold indicates a higher risk of developing irAE from ICI therapy. A TF threshold can be about 82.5%, or about 70, 75, 80, 81, 82, 82.5, 83, 85, or 90%. The risk can be predicted prior to a treatment with an ICI therapy. The method can further comprise administering to the subject having a lower risk of developing irAE an ICI therapy.

Classifying TCRβ genes can comprise: a) obtaining TCRβ genes sequences comprising multiple gene segments and somatic alterations; b) translating at least one of the multiple gene segments or somatic alterations into an amino acid sequence; c) identifying a TCRβ gene encoding an amino acid sequence capable of antigen recognition as a productive TCRβ gene, d) identifying a TCRβ gene without an amino acid sequence capable of antigen recognition as a non-productive TCRβ gene, e) repairing the amino acid sequence of a TCRβ gene identified as non-productive to generate a repaired TCRβ gene capable of antigen recognition, and f) classifying the TCRβ gene as a productive TCRβ genes or as a repaired TCRβ genes. The gene segments can be selected from the group consisting of variable (V) gene segments, diversity (D) gene segments, joining (J) gene segments, and any combination thereof. The non-productive TCRβ gene can be a TCRβ gene with out-of-frame gene segments or a TCRβ gene with a stop codon in a somatic junction between gene segments. Repairing non-productive TCRβ gene can comprise adding or removing one or more nucleotides at a somatic junction between gene segments to bring the gene segments in a same reading frame and/or mutating a nucleotide in a somatic region between gene segments to convert a stop codon into an amino acid. The TCRβ gene sequence can comprise a complimentary determining region 1 (CDR1) sequence of the TCRβ gene, a CDR2 sequence of the TCRβ gene, a CDR3 sequence of the TCRβ gene, a combination thereof, or a sequence of a complete TCRβ gene. The TCRβ gene sequence can comprise a CDR3 sequence of the TCRβ gene. The method can further comprise removing the first three amino acids and the last three amino acids of the CDR3 sequences from the TCRβ gene sequence. Obtaining a TCRβ gene sequence can comprise sequencing TCRβ genes from a peripheral blood mononucleated cell sample from the subject. Obtaining a TCRβ gene sequence can further comprise isolating T cells from the sample. Isolating T cells can be by cell sorting and/or RNA expression. T cells can be non-regulatory T cells.

The cell can be a peripheral blood mononucleated cell from a subject having cancer. The cancer can be selected from the group consisting of melanoma, prostate cancer, non-small cell lung cancer, mesothelioma, bladder cancer and renal cancer. The ICI therapy can be selected from the group consisting of ipilimumab, tremelimumab, atezolizumab, avelumab, durvalumab, nivolumab, pembrolizumab, ipilimumab plus nivolumab, and combinations thereof.

The compositions and methods are more particularly described below, and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### Example 1: Materials and Methods

### Clinical Data Sources and Collection

Clinical data from patients enrolled in an institutional prospective immunotherapy cohort, as well as from cases in three published studies were collected. As previously described, our cohort enrolled cancer patients planned for but not yet started on ICI. Data collected included demographics, tumor characteristics, treatment information, and irAE. Due to challenges in determining the occurrence, type, timing, and severity of irAE, two separate clinicians experienced in ICI administration and monitoring reviewed each case for toxicities, with discrepancies reviewed and adjudicated by a third experienced clinician. Enrolled patients underwent blood collection at pre-treatment baseline during ICI therapy. Published cohorts were selected for this analysis according to their publicly available clinical and biomarker data. For comparison, available T cell receptor sequencing data from healthy human subjects in a separate published study were used.

irAE were categorized as clinically significant (common terminology criteria for adverse events [CTCAE] grade ≥2) or not clinically significant (CTCAE grade ≤1). This threshold was chosen because, in general, grade 2 or greater toxicity implies need for medical intervention, whereas grade 1 toxicity is generally asymptomatic and requires neither ICI modification nor specific treatment. Prior studies have employed a similar cut-point to predict irAE after a single round of treatment.

### TCRβ Gene Sequencing

All TCRβ gene sequencing was performed by Adaptive Biotechnologies (Seattle, WA). For this study, the focus was on identifying an irAE predictor from unsorted T cells from peripheral blood samples. However, one of the study cohorts incorporated into the present analysis had sorted the T cells into CD4 and CD8 populations, with each subset sequenced separately. Because the ratio of TCRβ genes from the sorted CD4 and CD8 T cells was approximately 2:1 (the anticipated ratio of CD4 to CD8 T cells in peripheral blood), the TCRβ genes from the separately sequenced CD4 and CD8 T cell populations were merged into a single sample. This allowed us to compare the TCRβ genes from the sorted T cells to the unsorted T cells from the other studies.

### Single Cell Sequencing

Baseline peripheral blood samples from 5 cancer patients were subjected to single-cell RNA sequencing using the 10× genomics platform with libraries included to capture TCR sequences, including V(D)J recombination events. After collecting the sequences, we excluded all cells that did not have exactly one TRA and one TRB sequence per cell. We then measured CDR3a and CDR3b lengths.

### Productive and Non-Productive TCRβ Genes

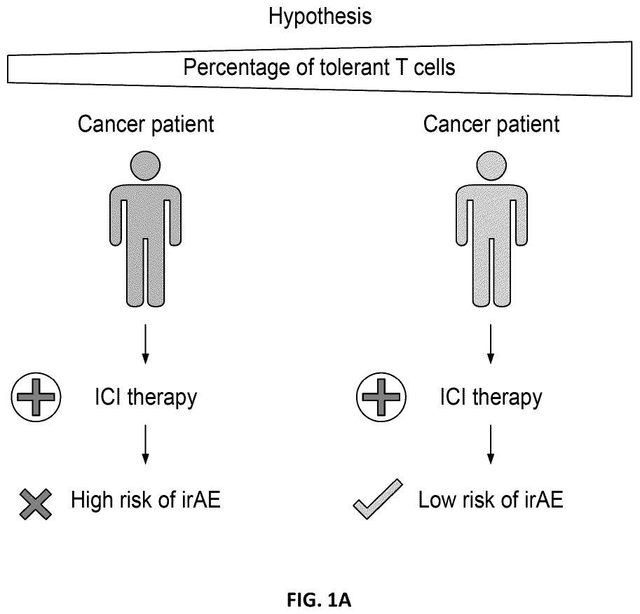

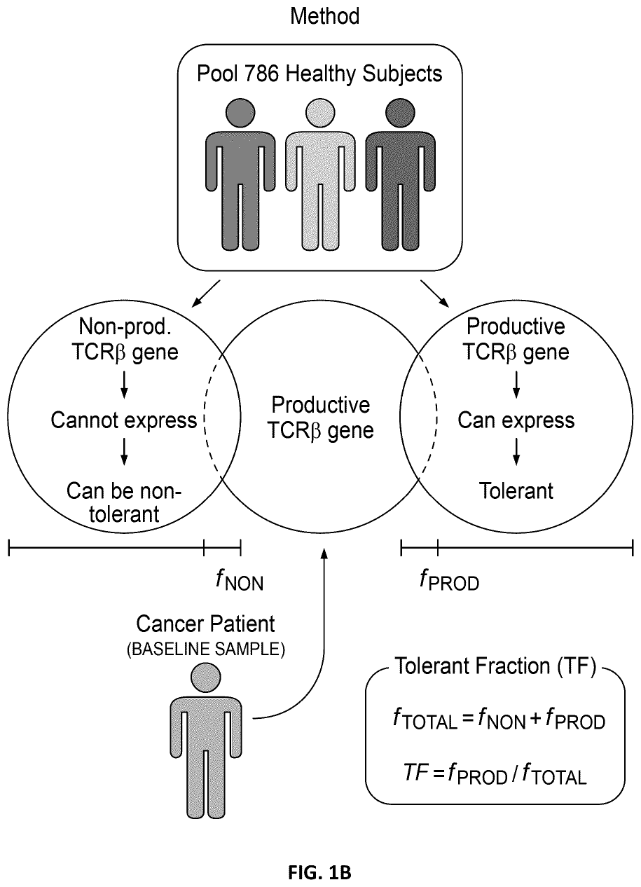

TRB gene sequencing reveals both productive and non-productive TRB genes in peripheral blood. Productive TRB genes, which we define as being in-frame and not containing a stop codon in the rearrangement, can express a TRB. Because the productive TRB genes can express a receptor, productive TRB genes are assumed to be tolerant (FIG. 1B). In contrast, non-productive TRB genes, which we define as being either out-of-frame or containing a stop codon in the rearrangement, cannot express a TRB. Because non-productive TRB genes cannot express a receptor, non-productive TRB genes cannot are not assumed to be necessarily tolerant (FIG. 1B)

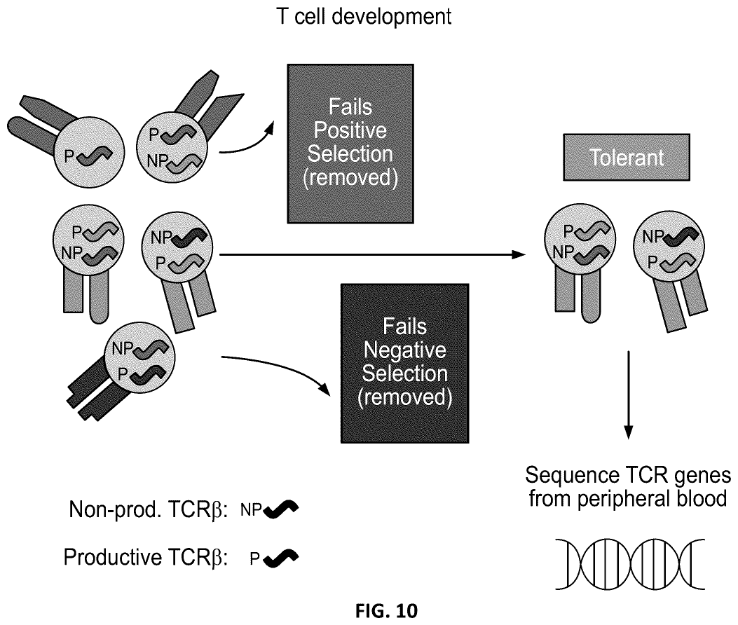

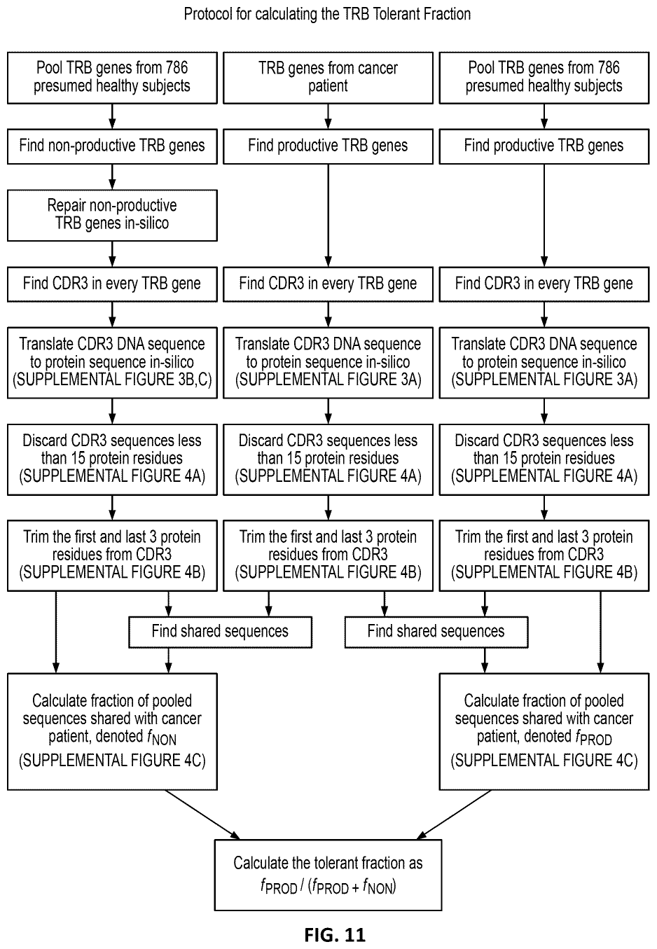

T cell selection, also known as thymic selection, represents the biological processes ensuring productive TRB sequences are tolerant (FIG. 10). During T cell selection, developing T cells can contain both a productive and non-productive TRB sequence on opposite chromosomes. However, only the productive TRB sequence can express TRB protein chains. Developing T cells expressing TRB chains that are not tolerant are deleted by T cell selection. Therefore, the surviving T cells are those that can express tolerant TRB protein chains. Because the expressed TRB protein chains must be tolerant, the productive TRB sequences expressing the TRB protein chains must also be tolerant. However, the non-productive TRB sequences are under no such constraint, and are carried through T cell selection without regard for whether the sequences are tolerant because the sequences do not express. After completing T cell selection, the surviving T cells enter peripheral blood, where the TRB sequences can be sequenced.

### Tolerant Fraction

To define parameters for T cell tolerance, non-productive and productive TCRβ genes from 786 subjects with no known disease were separate (FIG. 1B). The pool of non-productive TCRβ genes cannot express and therefore can be non-tolerant, while the pool of productive TCRβ genes can express and therefore are tolerant. Each cancer patient was characterized according to the relative overlap of their productive TCRβ genes with that of the tolerant pool (from the 786 subjects).

Specifically, f_{PROD} and f_{NON} were used to denote the fractions of tolerant and non-tolerant pools that overlap (the definition of overlap is provided later) with TCRβ genes from a cancer patient (FIG. 1B). The fraction of tolerant T cells, termed the tolerant fraction, was calculated for each cancer patient as follows:

$$TF {=}^{{f}_{PROD}} {/}_{{f}_{TOTAL}} ; {f}_{TOTAL} = {f}_{PROD} + {f}_{NON}$$

A value of 1 indicates all the TCRβ can assumed to be tolerant, while a value of 0 indicates none of the TCRβ can assumed to be tolerant.

The TRB sequences are subjected to in-silico processing and filtering steps before being used in the calculation of the tolerant fraction. These steps are summarized in FIG. 11 and described in the preceding sections.

### Translating TCRβ Genes to Protein Sequences

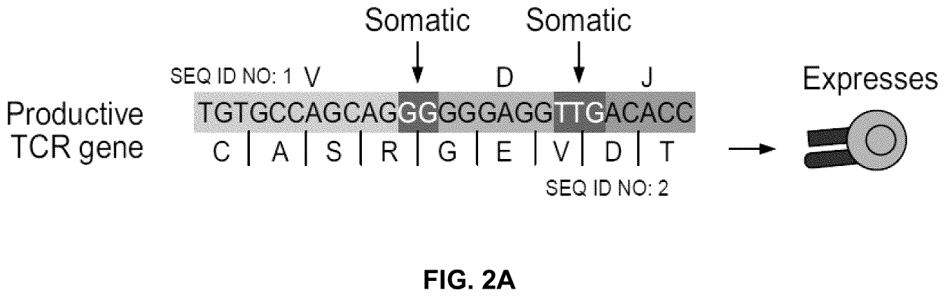

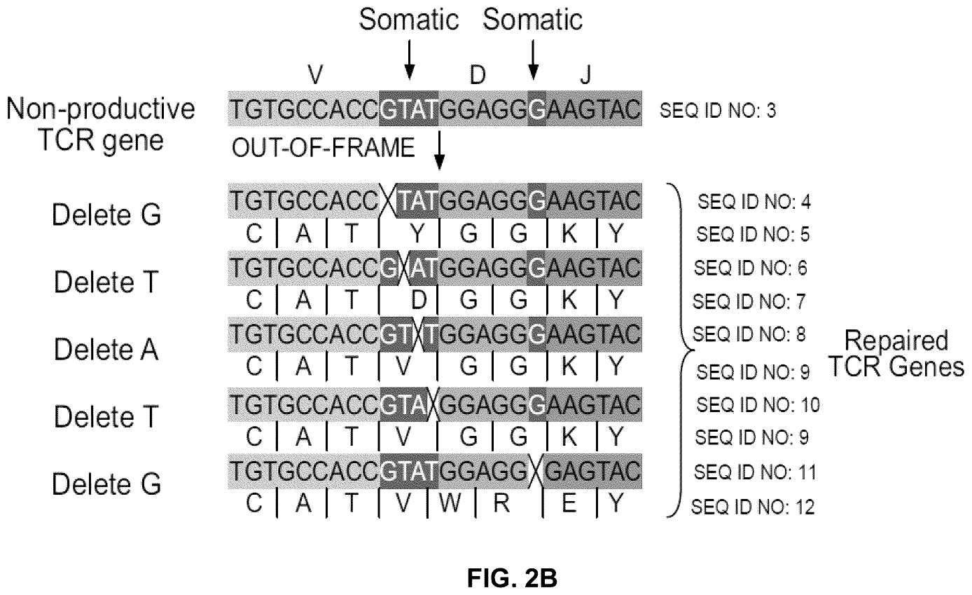

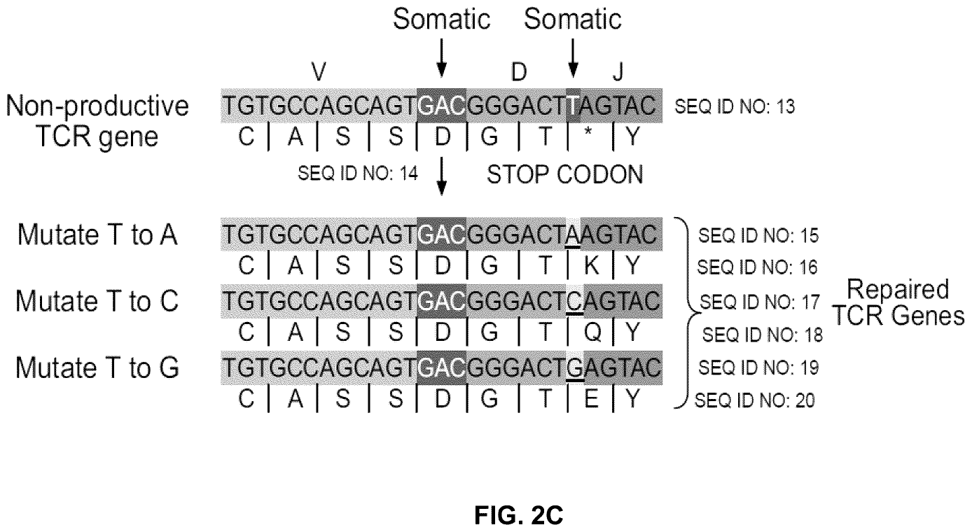

T cells specificity is based on the expressed TCR proteins, not the nucleotide sequences of the genes, motivating us to compare productive to non-productive TCR genes as protein sequences. Therefore, all TCRβ genes were translated in silico to protein sequences. While productive TCRβ genes can be translated to protein sequences, non-productive TCRβ genes cannot. An algorithm was previously developed to repair computationally non-productive TCRβ genes, thereby allowing repaired genes to be translated to productive protein sequences. To maximally preserve the original biological sequences, which contain complex and intricate biases from V(D)J recombination, our algorithm repairs each non-productive TCRβ gene using the fewest alterations required to obtain a productive copy (FIG. 2).

Our repairing algorithm, based on the type of repair required, handles each non-productive TRB gene as one of three cases.
1. For TRB genes that are non-productive because the open reading frame of the J segment is one position ahead of the open reading frame of the V segment, our algorithm removes (in silico) any single nucleotide at a somatic junction to bring the segments into the same open reading frame (FIG. 2B).
2. For TRB genes that are non-productive because the open reading frame of the J segment is two positions ahead of the open reading frame of the V segment, our algorithm removes (in silico) any two nucleotides at the somatic junctions to bring the segments into the same open reading frame.
3. For TRB genes that are non-productive because of a stop codon in a somatic junction, we mutate (in silico) any nucleotide in the somatic junction encoding the stop codon to attempt to convert it to an amino acid residue (FIG. 2C).

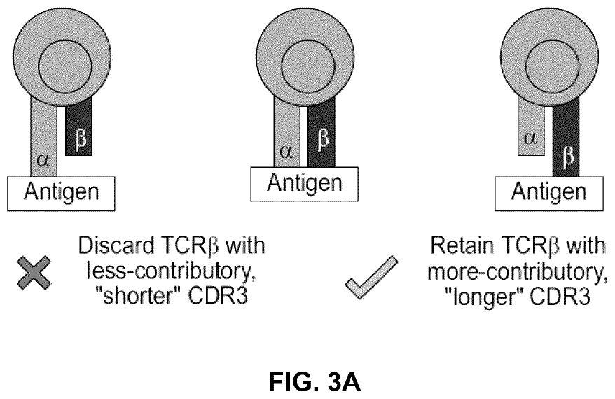

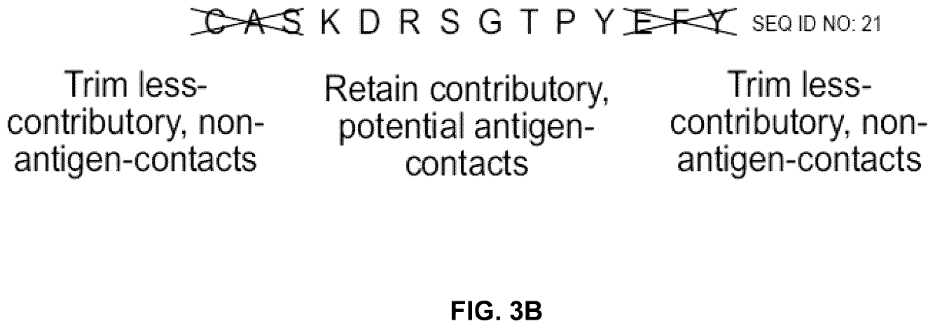

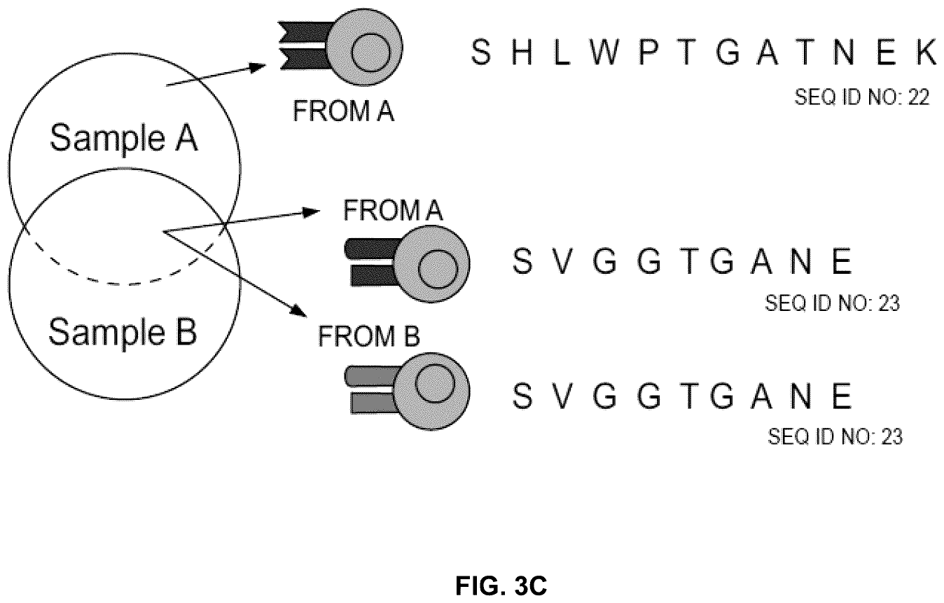

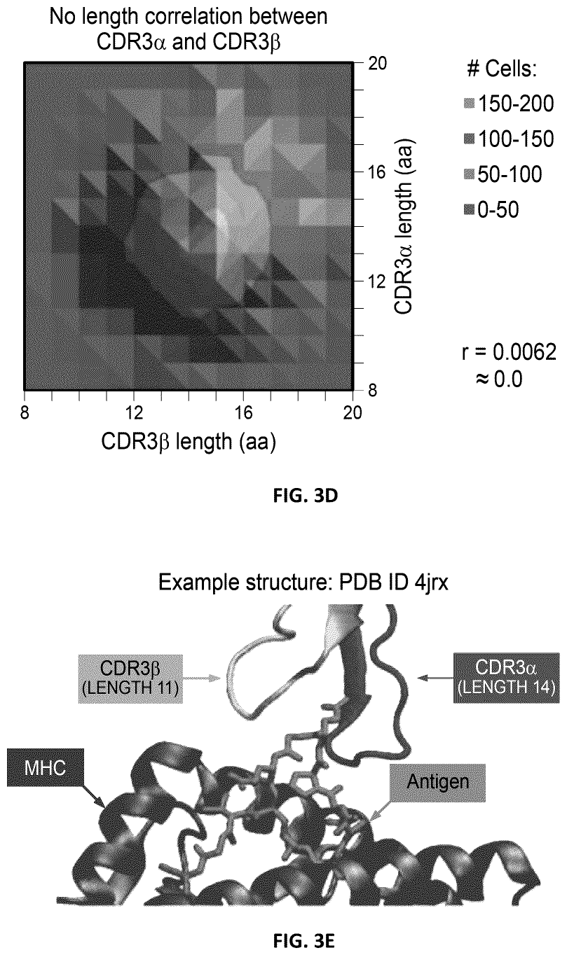

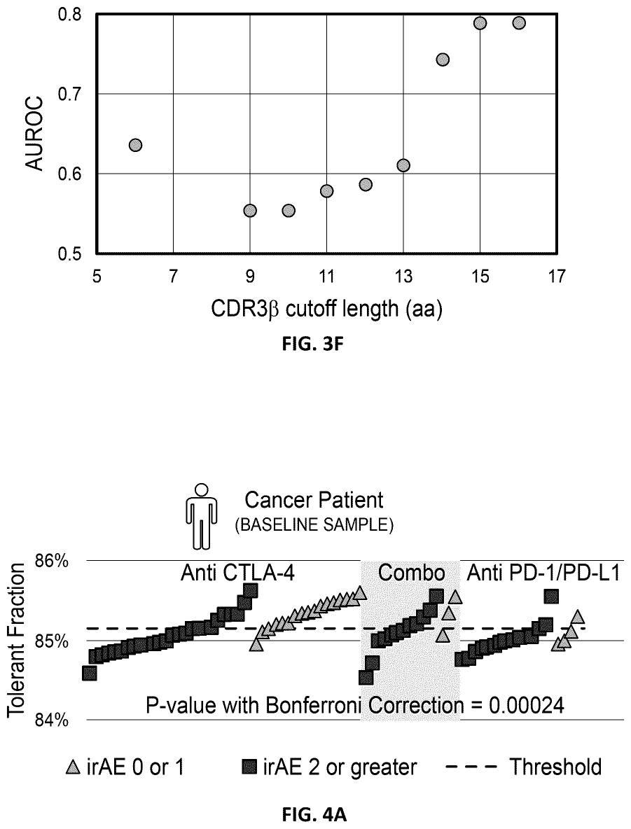

There are multiple ways to repair a non-productive TCRβ gene (FIG. 3). This study treats all protein sequences obtained from repairing the same non-productive TCRβ gene as valid. Therefore, all protein sequences obtained from repairing the same non-productive TCRβ gene are used in the calculation for the tolerant fraction.

Discarding TCRβ with “Short” TCRβ Sequences

A T cell receptor (TCR) is a heterodimer of a TCRα and TCRβ chain that each contribute a complimentary determining region 3 (CDR3) for antigen recognition. It was hypothesized the TCR chain with the longer CDR3 will contribute more to antigen recognition than the TCR chain with the shorter CDR3. Because only TCRβ sequences were available for this study, TCRβ sequences with a “short” CDR3 were discarded based on the assumption that a “short” CDR3 would not contribute to antigen recognition and therefore are not relevant to T cell tolerance (FIG. 3A). In this study, length cutoffs of 2, 9, 10, 11, 12, 13, 14, 15, and 16 as measured by the number of amino acid residues in CDR3 were considered. A cut-off of 15 amino acid residues was used for our analysis because it provided the best discrimination. All reported P-values are adjusted using a Bonferroni correction.

### Trimming CDR3 Sequences

Although the complimentary determining region 3 (CDR3) of each TCRβ is involved in antigen recognition, not all amino acid residues within the CDR3 make direct contact with antigens. Published analyses of 3D X-ray crystallographic structures of TCRβ in contact with antigen have demonstrated that the first and last three CDR3 amino acid residues do not directly contact antigen. Therefore, to determine the tolerant fraction, the first and last three amino acid residues from each CDR3 were removed, as they do not contribute to specificity and therefore would not be expected to contribute to tolerance (FIG. 2B).

### Definition of Overlap

In this study, the overlap between two sets of TCRβ genes was define as the region where TCRβ genes from each set have identical trimmed CDR3 sequences. The size of the overlapping region is not weighted by the template count of the TCRβ genes but is weighted by the number of times the same TCRβ chain (protein sequence) is found in different subjects (pooled from 786 subjects).

When calculating relative overlap, the template count of TRB sequences was not incorporated but did include duplicate TRB sequences in the tolerant and non-tolerant pools resulting from the aggregation of the 786 healthy subjects. It is also worth noting the template count of TRB sequences from the ICI-treated cancer patients (FIG. 1b) did not alter the calculation of relative overlap, which was verified by running the calculation with and without the template count of the TRB sequences from cancer patients.

### Statistical Analysis

Our null hypothesis is that the tolerant fraction of patients with a grade 0-1 irAE are not higher than the tolerant fraction of patients with a grade ≥2 irAE. The alternative hypothesis is that they are higher. Because we are not testing if they are lower, we use a one-sided test of our null hypothesis. Specifically, we calculate p-values using a one-sided Mann-Whitney U test assuming the null hypothesis. Because we considered multiple cutoffs for the CDR3 sequence lengths, we adjusted p-values using a Bonferroni correction.

T Cell Enrichment Against an irAE Antigen

Napsin-A is a potential T cell antigen that can help drive an irAE in patients with lung cancer. Starting with peripheral blood from four lung cancer patients, a recent study enriched for T cells specific for napsin A by co-culturing T cells with napsin A and then sorting for CD8 interferon (IFN)-γ-positive/tumor necrosis factor (TNF)-positive T cells.

TRB Sequences from Thymus and Peripheral Blood Samples

To evaluate the tolerant fraction further, publicly available TRB sequences were analyzed from matched thymus and peripheral blood samples from pediatric patients undergoing corrective cardiac surgery and then made publicly available. The samples were subjected to TRB sequencing by Adaptive Biotechnologies (Seattle, WA), and the results were downloaded to calculate the tolerant fraction of these TRB sequences. Because the thymus is enriched with developing T cells not yet removed by T cell selection, we hypothesized that thymus samples would have a lower tolerant fraction than peripheral samples.

### Disease-Specific TRB Sequences

TRB sequences from various clinical disease scenarios, including infection (influenza, coronavirus), and autoimmune diseases (type 1 diabetes mellitus [T1DM] and multiple sclerosis [MS]) were also examined. For these analyses, TRB sequences did not come from individual patients, but were instead curated from pooled published literature available through the Immune Epitope Database (IEDB). In general, each study from the pool of publications contributes a small number of TRB sequences, with TRB sequences being sourced from tissue samples and peripheral blood using a diverse array of experimental methodologies. For each clinical disease condition, we aggregated TRB sequences, which allowed calculation of the tolerant fraction.

### Example 2: Results

A total of 77 patients (including 22 from our institutional cohort and 55 from three published studies) were analyzed in this study (Table 1). ICI types included anti-CTLA4 (n=43), anti-PD1/PDL1 (n=19), and combination anti-CTLA4 plus anti-PD1/PDL1 (n=15) as follows: ipilimumab (n=22), tremelimumab (n=21), atezolizumab (n=2), avelumab (n=1), durvalumab (n=2), nivolumab (n=9), pembrolizumab (n=5), and ipilimumab plus nivolumab (n=15). Cancer types included the following melanoma (n=45), prostate cancer (n=10), non-small cell lung cancer (n=4), small cell lung cancer (n=1), mesothelioma (n=1), bladder cancer (n=1), and renal cell carcinoma (n=1). Overall, 53 patients (68.8%) had a grade ≥2 irAE; 24 patients had grade 0-1 irAE (31.2%).

TABLE 1
Source of the patients. This study includes patients with baseline samples
from three published studies as well as patients from this study. The table
shows the study, number of patients, type of cancer, and ICI therapy.

| Study | Patients | Cancer | Treatment |
|---|---|---|---|
| Robert, Clinical | 21 | Melanoma | Tremelimumab |
| Cancer Research, | | | |
| 2014 | | | |
| Subudhi, PNAS, 2016 | 19 | Prostate Cancer | Ipilimumab |
| Lozano, Nature | 15 | Melanoma | Ipilimumab and Nivolumab |
| Medicine, 2022 | | | |
| This Study | 22 | Bladder Cancer, Renal | Atezolizumab, Avelumab, |
| | | Cell Cancer, | Durvalumab, Ipilimumab, |
| | | Mesothelioma, | Nivolumab, or |
| | | Melanoma, NSCLC, | Pembrolizumab |
| | | SCLC | |

To assess our hypothesis that longer TCR CDR3 are most involved in antigen interactions and therefore most relevant to autoimmune phenomena including irAE, we examined various characteristics of TCR length (FIG. 3). We found no significant correlation between CDR3a and CDR3b lengths within individual cells (Pearson correlation coefficient r=0.0062) (FIG. 3D). FIG. 3E demonstrates a representative 3-dimensional x-ray crystallographic structure in which the longer TCR chain achieves greater antigen contact (≈55% of amino acid residues) than does the shorter TCR chain (≈25% of residues). We then considered TCR chain length cutoffs of 2, 9, 10, 11, 12, 13, 14, 15, and 16 amino acid residues (FIG. 3F), observing a clear association between higher TCR chain length cutoff and performance of the tolerant fraction to predict irAE. A length of 15 amino acid residues provided the best discrimination and was therefore selected as a cut-off.

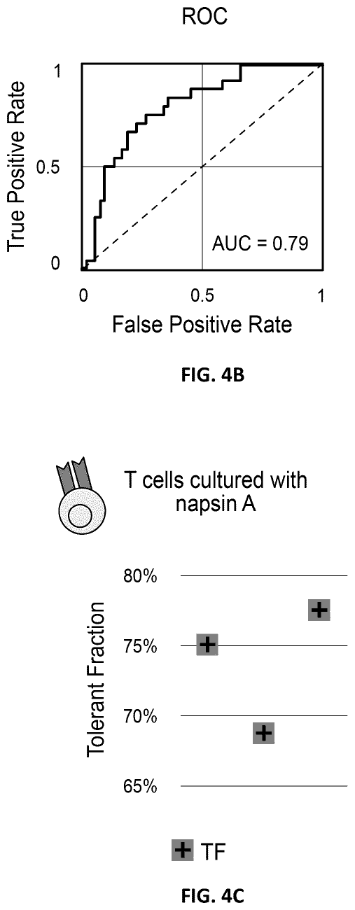

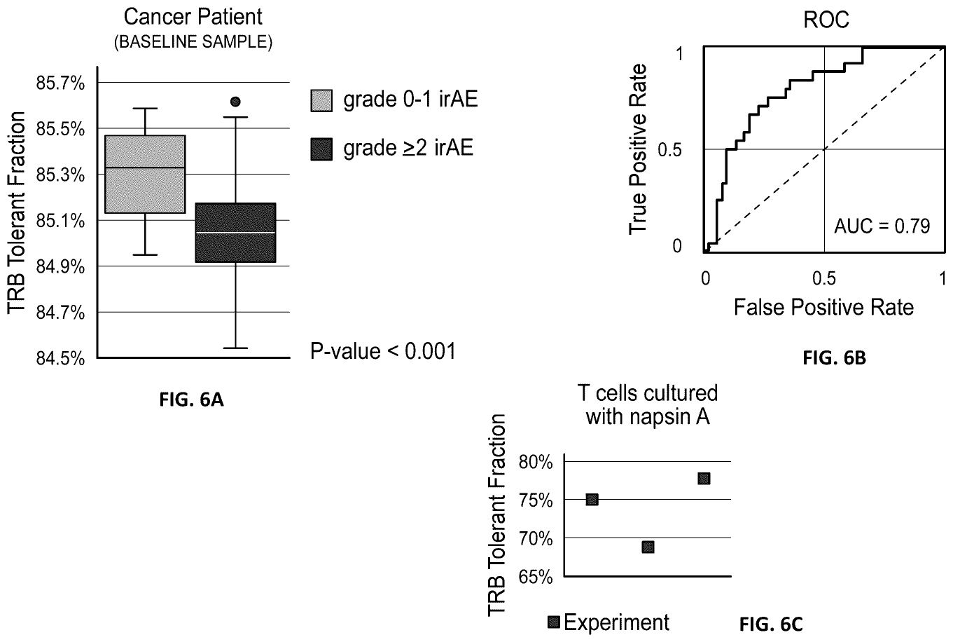

To evaluate the hypothesis that baseline T cell tolerance is associated with lower risk of autoimmune toxicity, the TCRβ tolerant fraction was measured in patients with and without clinically significant irAE (FIGS. 4A and 6A). As hypothesized, tolerant fraction values were higher in patients with grade 0-1 irAE compared than in patients with grade ≥2 irAE (P<0.001). Among patients with grade 0-1 irAE, 18 of 24 had a tolerant fraction >85.2% (75% sensitivity). Among patients with grade ≥2 irAE, 39 of 53 had a tolerant fraction <85.2% (74% specificity). The predictive ability of tolerant fraction appeared stronger for anti-CTLA-4 and anti-CTLA4 plus anti-PD1/PDL1 combination therapy than for PD-1/PD-L1 monotherapy (FIGS. 4A and 6A). As shown in FIGS. 4B and 6B, for all possible cut-offs for tolerant fraction values, the true positive rate was almost always greater than the false positive rate. The area under the curve (AUC) of the receiver operator characteristics (ROC) was 0.79 (FIGS. 4B and 6B).

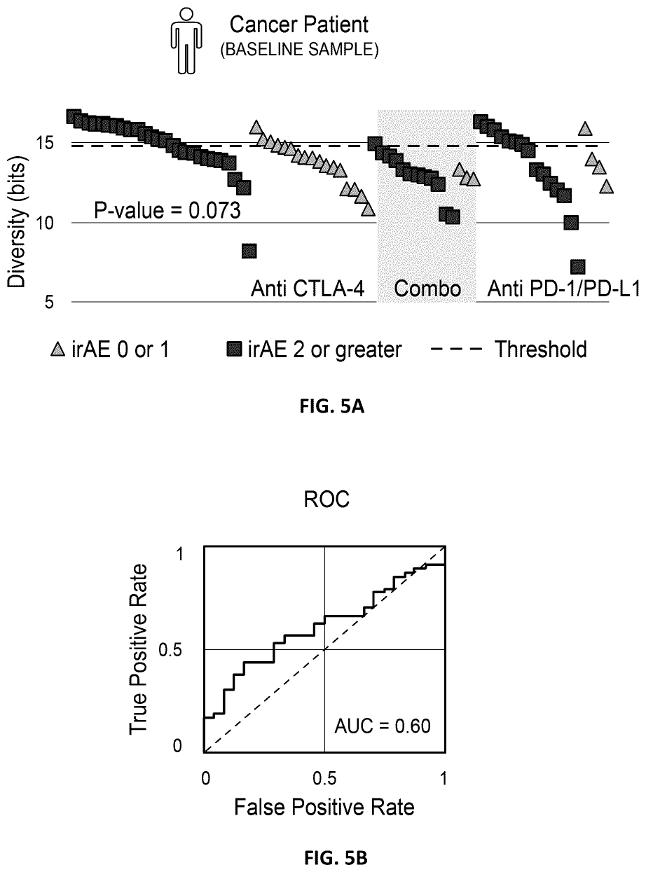

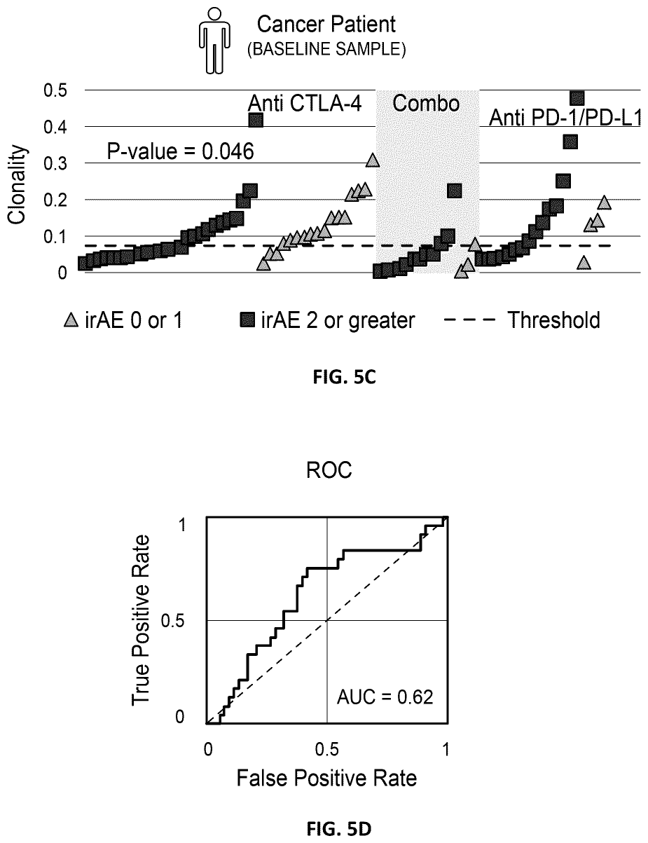

The tolerant fraction for T cells capable of recognizing antigens associated with irAE was also determined. For this analysis, the tolerant fraction of T cells enriched against Napsin A was measured. Napsin A is an antigen expressed in over 80% of lung adenocarcinoma cases, and T cells enriched against Napsin A have been associated with lung inflammation driving pulmonary irAE. Tolerant fraction values for three samples enriched against napsin A were 75.0%, 68.8%, and 77.6% (FIGS. 4C and 5C), falling well in the range (<82.5%) associated with irAE (the tolerant fraction for a fourth sample could not be calculated because there is no overlap between the fourth sample and the TCRβ genes from the 786 subjects).

To place the findings in context of other predictive parameters, TCRβ diversity and clonality in the instant 77 cases were also evaluated. For TCRβ diversity, ROC AUC was 0.60 (P=0.073) (FIG. 5 A, 5B). For TCRβ clonality, ROC AUC was 0.62 (P=0.046) (FIG. 5C, 5D).

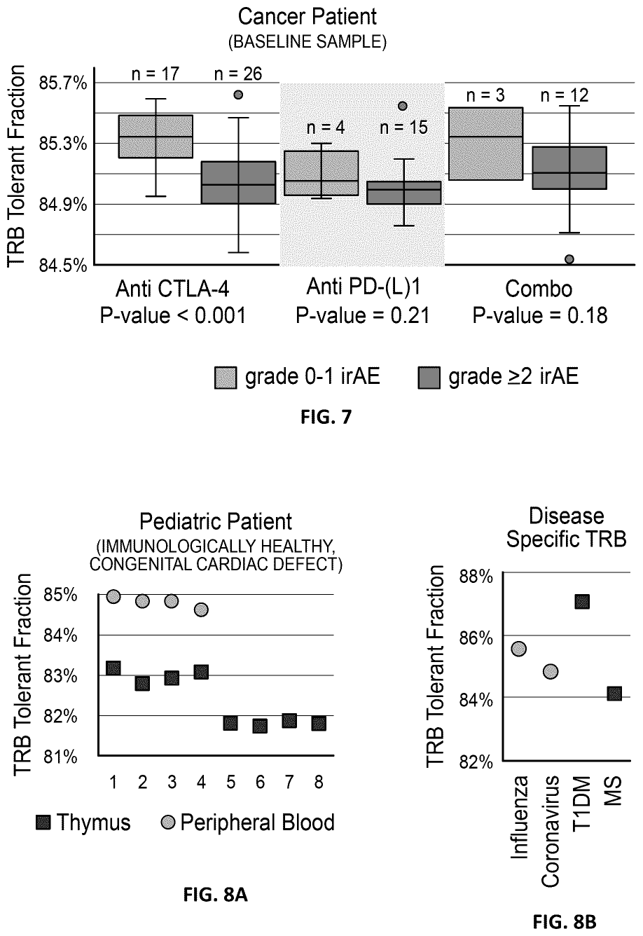

The predictive performance of the tolerant fraction for specific ICI treatment types (CTLA4, PD1/PDL1, combination CTLA4+PD1/PDL1) was also examined (FIG. 7). For all three categories, the tolerant fraction was lower in cases grade ≥2 irAE. This difference reached statistical significance for CTLA4 (n=43; P<0.001) and showed non-significant trends for PD1/PDL1 (n=19; P=0.21) and combination CTLA4+PD1/PDL1 (n=15; P=0.18).

To evaluate the TRB tolerant fraction in various clinical disease states, publicly available individual patient and pooled TRB sequences were examined (FIG. 8). In an immunologically healthy population of pediatric patients undergoing surgery for congenital cardiac defects, matched thymus and peripheral samples demonstrated substantially lower TRB tolerant fractions for thymic TRB sequences (FIG. 8A). TRBs specific for infection (influenza and coronavirus) demonstrated a tolerant fraction in the 85-86% range, while TRBs specific for T1DM had a higher tolerant fraction (≈87%), and TRBs specific for MS had a lower tolerant fraction (≈84%) (FIG. 8B).

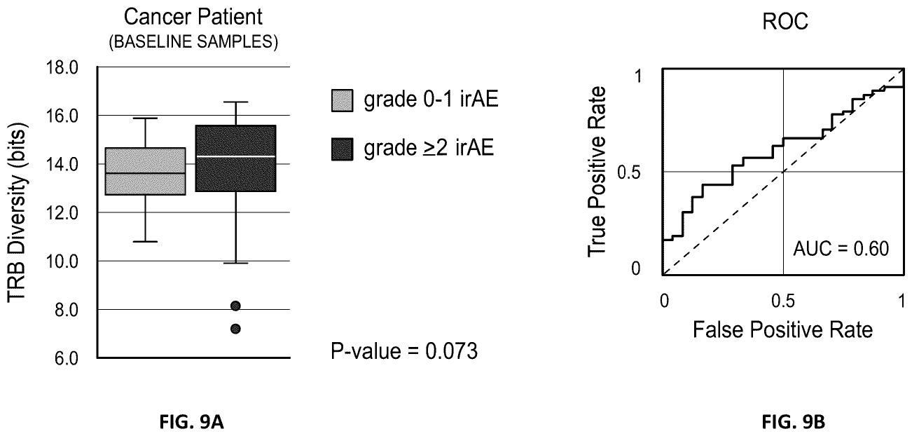

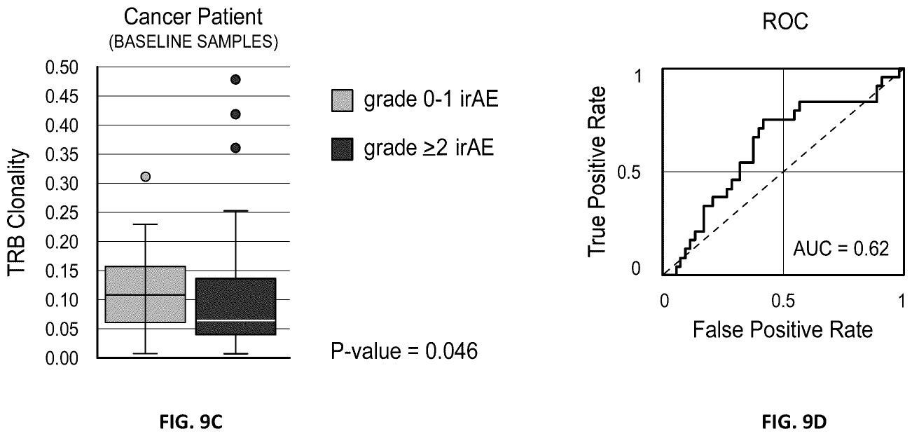

To place the findings in context of other predictive parameters, we also evaluated TRB diversity and clonality in our 77 cases of ICI-treated cancer patients (FIG. 9). For TRB diversity, the ROC AUC was 0.60 (P=0.07) (FIGS. 9A-9B). For TRB clonality, the ROC AUC was 0.62 (P=0.05) (FIGS. 9C-9D).

These data indicate:
(i) patients with MS have a worse (lower) tolerant fraction than patients without the disease indicating that the methods described above can be used for diagnosing or prognosticating MS
(ii) patients with covid have a slightly worse (lower) tolerant fraction than patients with influenza indicating that the methods described above can be used for diagnosing or prognosticating long covid

### Example 3: Discussion

Immune-related adverse events remain a major concern in immuno-oncology. These autoimmune toxicities may affect almost any organ system. In rare cases, they may be permanent or even fatal. The lack of understanding of these clinical phenomena is apparent through the relatively blunt approach to patient selection and monitoring. Apart from the observation that patients with pre-existing autoimmune disease may face heightened risk of autoimmune disease flare and irAE, and patients with organ transplant may face risk of organ rejection, there remains no clear method to identify high-risk populations. Similarly, recommendations for irAE monitoring range from following only thyroid, liver, and renal function to extensive panels including these parameters as well as assessment of cardiac, pulmonary, pituitary, adrenal, and pancreatic function.

In response to a clear clinical need, characterization of patient T cell function through such parameters as TCR diversity and clonality has emerged as a potential approach to irAE prediction. Premised on the tenet that tolerance suppresses anti-self-immunity, in the present study a new T cell metric—the tolerant fraction—is proposed as a potential means to identify patients at heightened risk for future irAE. With this approach, a specific tolerant fraction threshold (e.g., 82.5%) was identified as able to distinguish between low- and high-risk of future irAE in a cohort comprising both published and unpublished TCR and clinical data from cases with diverse cancer types treated with various types of immunotherapies. Consistent with the overall concept of tolerance, patients with more tolerant T cells, manifest as a higher tolerant fraction, were less likely to develop clinically significant irAE. With an ROC AUC approximately 0.8, in the present study the tolerant fraction had superior performance to both TCR diversity and clonality (ROC AUC approximately 0.6) for irAE prediction. It was also noted a clear association between low tolerance and presence of T cell populations enriched against irAE-associated antigens.

The association between immune tolerance and irAE is consistent with earlier observations linking immune dysregulation with immunotherapy toxicity. Specifically, a signature featuring low baseline but marked increases in IFNγ-inducible cytokines/chemokines involved in T cell recruitment and activation was associated with heightened risk of future irAE. Clinical correlates of this relationship include the subset of primary immunodeficiencies termed disease of immune dysregulation, in which patients with impaired immunity develop autoimmune and inflammatory disorders. Similar observations have been made in cases of acquired immunodeficiency such as HIV.

Potential clinical application of tolerant fraction requires consideration of certain factors. First, the relatively narrow range between the extremes of tolerant fraction values in the cohort. A potential explanation for this observation is that only a small fraction of pre-treatment T cells are mechanistically associated with subsequent irAE. Better predictive performance of tolerant fraction among patients treated with anti-CTLA4-containing regimens rather than exclusively PD1/PDL1-based treatments are also needed. Some patients in the present study received prior chemotherapy, which could affect T cell populations.

Strengths of the current study include the total number of patients, which exceeds those in a number of previously published studies of TCR-based irAE correlative biomarkers. This study also included diverse cancer and ICI treatment types. From a clinical perspective, the T cell tolerant fraction has the favorable characteristic of being a pre-treatment baseline parameter. In contrast, assessment of T cell clonal expansion or diversification requires assessment of immune cell populations over time. Indeed, the availability of pre-treatment guidance could be particularly helpful for those areas of greatest current need: selection of patients and monitoring.

In conclusion, a novel parameter, the T cell tolerant fraction has been identified, which is associated with future development of clinically significant irAE. Unlike dynamic T cell clonal expansion or diversification, the tolerant fraction may be determined prior to ICI initiation, thereby informing up-front patient selection and monitoring. Furthermore, in the present study cohort, tolerant fraction has better predictive ability than pre-treatment TCR clonality or diversity.

### REFERENCES

Y. Jiang, N. Zhang, H. Pang, X. Gao and H. Zhang, “Risk and incidence of fatal adverse events associated with immune checkpoint inhibitors: a systematic review and meta-analysis,” Therapeutics and Clinical Risk Management, vol. 15, pp. 293-302, 2019.
D. Hsiehchen, M. K. Watters, R. Lu, Y. Xie and D. E. Gerber, “Variation in the assessment of immune-related adverse event occurrence, grade, and timing in patients receiving immune checkpoint inhibitors,” JAMA network open, vol. 2, no. 9, pp. e1911519-e1911519, 2019.
S. K. Subudhi, A. Aparicio, J. Gao, A. J. Zurita, J. C. Araujo, C. J. Logothetis, S. A. Tahir, B. R. Korivi, R. S. Slack and L. Vence, “Clonal expansion of CD8 T cells in the systemic circulation precedes development of ipilimumab-induced toxicities,” Proceedings of the National Academy of Sciences, vol. 113, no. 42, pp. 11919-11924, 2016.
D. Y. Oh, J. Cham, L. Zhang, G. Fong, S. S. Kwek, M. Klinger, M. Faham and L. Fong, “Immune Toxicities Elicted by CTLA-4 Blockade in Cancer Patients Are Associated with Early Diversification of the T-cell Repertoirelpilimumab-Induced T-cell Diversity Predicts IRAEs,” Cancer research, vol. 77, no. 6, pp. 1322-1330, 2017.
A. X. Lozano, A. A. Chaudhuri, A. Nene, A. Bacchiocchi, N. Earland, M. D. Vesely, A. Usmani, B. E. Turner, C. B. Steen and B. A. Luca, “T cell characteristics associated with toxicity to immune checkpoint blockade in patients with melanoma,” Nature medicine, vol. 28, no. 2, pp. 353-362, 2022.
L. Robert, J. Tsoi, X. Wang, R. Emerson, B. Homet, T. Chodon, S. Mok, R. R. Huang, A. J. Cochran and B. Comin-Anduix, “CTLA4 Blockade Broadens the Peripheral T-Cell Receptor RepertoireCTLA4 Blockade Broadens the Peripheral TCR Repertoire,” Clinical Cancer Research, vol. 20, no. 9, pp. 2424-2432, 2014.
M. S. von Itzstein, A. S. Gonugunta, T. Sheffield, J. Homsi, J. E. Dowell, A. Y. Koh, P. Raj, F. Fattah, Y. Wang and V. S. Basava, “Association between Antibiotic Exposure and Systemic Immune Parameters in Cancer Patients Receiving Checkpoint Inhibitor Therapy,” Cancers, vol. 14, no. 5, p. 1327, 2022.
S. Khan, S. A. Khan, X. Luo, F. J. Fattah, J. Saltarski, Y. Gloria-McCutchen, R. Lu, Y. Xie, Q. Li and E. Wakeland, “Immune dysregulation in cancer patients developing immune-related adverse events,” British journal of cancer, vol. 120, no. 1, pp. 63-68, 2019.
R. O. Emerson, W. S. DeWitt, M. Vignali, J. Gravley, J. K. Hu, E. J. Osborne, C. Desmarais, M. Klinger, C. S. Carlson, J. A. Hansen, M. Rieder and H. S. Robins, “Immunosequencing identifies signatures of cytomegalovirus exposure history and HLA-mediated effects on the T cell repertoire,” Nature Genetics, vol. 49, no. 5, pp. 659-665, 2017.
NCI, “Common Terminology Criteria for Adverse Events (CTCAE),” [Online]. Available: ctep.cancer.gov/protocoldevelopment/electronic_applications/ctc.htm. [Accessed 9 2022].
F. Berner, D. Bomze, C. Lichtensteiger, V. Walter, R. Niederer, O. Hasan Ali, N. Wyss, J. Bauer, L. K. Freudenmann and A. Marcu, “Autoreactive napsin A-specific T cells are enriched in lung tumors and inflammatory lung lesions during immune checkpoint blockade,” Science immunology, vol. 7, no. 75, p. eabn9644, 2022.
C. S. Carlson, R. O. Emerson, A. M. Sherwood, C. Desmarais, M.-W. Chung, J. M. Parsons, M. S. Steen, M. A. LaMadrid-Herrmannsfeldt, D. W. Williamson and R. J. Livingston, “Using synthetic templates to design an unbiased multiplex PCR assay,” Nature communications, vol. 4, no. 1, pp. 1-9, 2013.
B. J. Manfras, D. Terjung and B. O. Boehm, “Non-productive human TCR β chain genes represent V-D-J diversity before selection upon function: insight into biased usage of TCRBD and TCRBJ genes and diversity of CDR3 region length,” Human Immunology, vol. 60, no. 11, pp. 1090-1100, 1999.
J. Ostmeyer, L. Cowell, B. Greenberg and S. Christley, “Reconsituting T Cell Receptor Selection In-silico,” Genes & Immunity, 2021.
H. Li, C. Ye, G. Ji, X. Wu, Z. Xiang, Y. Li and et al., “Recombinatorial Biases and Convergent Recombination Determine Interindividual TCRβ Sharing in Murine Thymocytes,” Journal of Immunology, vol. 189, no. 5, pp. 2404-2413, 2012.
N. Heikkilä, R. Vanhanen, D. A. Yohannes, I. Kleino, I. P. Mattila, J. Saramaki and et al., “Human thymic T cell repertoire is imprinted with strong convergence to shared sequences,” Molecular Immunology, vol. 127, pp. 112-123, 2020.
L. Ott de Bruin, M. Bosticardo, A. Barbieri, S. Lin, J. Rowe, P. Poliani, K. Ching, D. Eriksson, N. Landegren, O. Kämpe, J. Manis and N. LD, “Hypomorphic Rag1 mutations alter the preimmune repertoire at early stages of lymphoid development,” Blood, vol. 132, no. 3, pp. 281-292, 2018.
C. Pannetier, M. Cochet, S. Darche, A. Casrouge, M. Zoller and P. Kourilsky, “The sizes of the CDR3 hypervariable regions of the murine T-cell receptor beta chains vary as a function of the recombined germ-line segments,” Proceedings of the National Academy of Sciences of the United States of America, vol. 90, no. 9, pp. 4319-4323, 1993.
T. Funck, M. B. Barnkob, N. Holm, L. Ohm-Laursen, C. S. Mehlum, S. Möller and et al., “Nucleotide Composition of Human Ig Nontemplated Regions Depends on Trimming of the Flanking Gene Segments, and Terminal Deoxynucleotidyl Transferase Favors Adding Cytosine, Not Guanosine, in Most VDJ Rearrangements,” Journal of Immunology, vol. 201, no. 6, pp. 1765-1774, 2018.
E. Q. Roldan, A. Sottini, A. Bettinardi, A. Albertini, L. Imberti and D. Primi, “Different TCRBV genes generate biased patterns of V-D-J diversity in human T cells,” Immunogenetics, vol. 41, no. 2, pp. 91-100, 1995.
S. K. Srivastava and H. S. Robins, “Palindromic Nucleotide Analysis in Human T Cell Receptor Rearrangements,” PLOS ONE, vol. 7, no. 12, 2012.
J. Glanville, H. Huang, A. Nau, O. Hatton, L. E. Wagar, F. Rubelt, X. Ji, A. Han, S. M. Krams, C. Pettus, N. Haas, C. S. L. Arlehamn, A. Sette, S. D. Boyd, T. J. Scriba, O. M. Martinez and M. M. Davis, “Identifying specificity groups in the T cell receptor repertoire,” Nature, vol. 547, no. 7661, pp. 94-98, 2017.
J. Ostmeyer, S. Christley, I. T. Toby and L. G. Cowell, “Biophysicochemical Motifs in T-cell Receptor Sequences Distinguish Repertoires from Tumor-Infiltrating Lymphocyte and Adjacent Healthy Tissue,” Cancer Research, vol. 79, no. 7, pp. 1671-1680, 2019.
N. G. Ordóñez, “Napsin A expression in lung and kidney neoplasia: a review and update,” Advances in anatomic pathology, vol. 19, no. 1, pp. 66-73, 2012.
M. S. Von Itzstein, A. S. Gonugunta, H. G. Mayo, J. D. Minna and D. E. Gerber, “Immunotherapy use in patients with lung cancer and comorbidities,” Cancerjournal (Sudbury, Mass.), vol. 26, no. 6, p. 525, 2020.
J. R. Brahmer, H. Abu-Sbeih, P. A. Ascierto, J. Brufsky, L. C. Cappelli, F. B. Cortazar, D. E. Gerber, L. Hamad, E. Hansen and D. B. Johnson, “Society for Immunotherapy of Cancer (SITC) clinical practice guideline on immune checkpoint inhibitor-related adverse events,” Journal for immunotherapy of cancer, vol. 9, no. 6, 2021.
J. R. Brahmer, C. Lacchetti, B. J. Schneider, M. B. Atkins, K. J. Brassil, J. M. Caterino, I. Chau, M. S. Ernstoff, J. M. Gardner and P. Ginex, “Management of immune-related adverse events in patients treated with immune checkpoint inhibitor therapy: American Society of Clinical Oncology Clinical Practice Guideline,” Journal of clinical oncology: official journal of the American Society of Clinical Oncology, vol. 36, no. 17, p. 1714, 2018.
I. Puzanov, A. Diab, K. Abdallah, C. 3. Bingham, 3. Brogdon, R. Dadu, L. Hamad, S. Kim, M. Lacouture and N. LeBoeuf, “Managing toxicities associated with immune checkpoint inhibitors: consensus recommendations from the Society for Immunotherapy of Cancer (SITC) Toxicity Management Working Group,” Journal for immunotherapy of cancer, vol. 5, no. 1, pp. 1-28, 2017.
O. Khan and J. Monzon, “Diagnosis, monitoring, and management of adverse events from immune checkpoint inhibitor therapy,” Current Oncology, vol. 27, no. s2, pp. 43-50, 2020.
J. Haanen, F. Carbonnel, C. Robert, K. Kerr, S. Peters, J. Larkin and K. Jordan, “Management of toxicities from immunotherapy: ESMO Clinical Practice Guidelines for diagnosis, treatment and follow-up,” Annals of oncology, vol. 28, pp. iv119-iv142, 2017.
G. Azizi, M. R. Pouyani, H. Abolhassani, L. Sharifi, J. Mohammadi, A. Mirshafiey and A. Aghamohammadi, “Cellular and molecular mechanisms of immune dysregulation and autoimmunity,” Cellular immunology, vol. 310, pp. 14-26, 2016.
G. Zandman-Goddard and Y. Shoenfeld, “HIV and autoimmunity,” Autoimmunity reviews, vol. 1, no. 6, pp. 329-337, 2002.

Although the invention has been described with reference to the above examples, it will be understood that modifications and variations are encompassed within the spirit and scope of the invention. Accordingly, the invention is limited only by the following claims.

What is claimed is:

1. A method of predicting a risk of developing an immune-related adverse event (irAE) from an immune checkpoint inhibitor (ICI) therapy in a subject comprising:
a) classifying T cell receptor β (TCRβ) genes of a cell from the subject as productive TCRβ gene or repaired TCRβ gene;
b) classifying TCRβ genes of a cell from a pool of donors as a productive TCRβ gene or repaired TCRβ gene; and
c) calculating a tolerant fraction (TF) score, wherein the TF score is a ratio of a productive fraction (F_{PROD}) and a total fraction (F_{TOTAL}), wherein F_{PROD} is the number of TCRβ genes identified as productive TCRβ genes from the subject that overlap with TCRβ genes identified as productive TCRβ genes from the pool of donors, and F_{TOTAL} is sum of (i) the number of TCRβ genes identified as productive TCRβ genes from the subject that overlap with TCRβ genes identified as productive TCRβ genes from the pool of donors and (ii) the number of TCRβ genes identified as productive TCRβ genes from the subject that overlap with TCRβ genes identified as repaired TCRβ genes from the pool of donors, thereby predicting a risk of developing irAE from ICI therapy.

2. The method of claim 1, wherein productive TCRβ genes are likely to produce a T cell receptor (TCR) that is tolerant to self-antigens.

3. The method of claim 1, wherein repaired TCRβ genes are likely to produce a TCR that is not tolerant to self-antigens.

4. The method of claim 3, wherein TCRs that are not tolerant to self-antigens are likely to induce auto-immune toxicity.

5. The method of claim 1, further comparing the TF score to a TF threshold, wherein a TF score greater than the TF threshold indicates a lower risk of developing irAE from ICI therapy and a TF score lower than the TF threshold indicates a higher risk of developing irAE from ICI therapy.

6. The method of claim 5, wherein the TF threshold is about 82.5%.

7. The method of claim 1, wherein the risk is predicted prior to a treatment with an ICI therapy.

8. The method of claim 1, further comprising administering to the subject having a lower risk of developing irAE an ICI therapy.

9. The method of claim 1, wherein classifying TCRβ genes comprises:
a) obtaining TCRβ genes sequences comprising multiple gene segments and somatic alterations;
b) translating at least one of the multiple gene segments or somatic alterations into an amino acid sequence;
c) identifying a TCRβ gene encoding an amino acid sequence capable of antigen recognition as a productive TCRβ gene,
d) identifying a TCRβ gene without an amino acid sequence capable of antigen recognition as a non-productive TCRβ gene,
e) repairing the amino acid sequence of a TCRβ gene identified as non-productive to generate a repaired TCRβ gene capable of antigen recognition, and
f) classifying the TCRβ gene as a productive TCRβ genes or as a repaired TCRβ genes.

10. The method of claim 9, wherein the gene segments are selected from the group consisting of variable (V) gene segments, diversity (D) gene segments, joining (J) gene segments, and any combination thereof.

11. The method of claim 9, wherein the non-productive TCRβ gene is a TCRβ gene with out-of-frame gene segments or a TCRβ gene with a stop codon in a somatic junction between gene segments.

12. The method of claim 9, wherein repairing non-productive TCRβ gene comprises adding or removing one or more nucleotides at a somatic junction between gene segments to bring the gene segments in a same reading frame and/or mutating a nucleotide in a somatic region between gene segments to convert a stop codon into an amino acid.

13. The method of claim 9, wherein the TCRβ gene sequence comprises a complimentary determining region 1 (CDR1) sequence of the TCRβ gene, a CDR2 sequence of the TCRβ gene, a CDR3 sequence of the TCRβ gene, a combination thereof, or a sequence of a complete TCRβ gene.

14. The method of claim 9, wherein the TCRβ gene sequence comprises a CDR3 sequence of the TCRβ gene.

15. The method of claim 14, further comprising removing the first three amino acids and the last three amino acids of the CDR3 sequences from the TCRβ gene sequence.

16. The method of claim 9, wherein obtaining a TCRβ gene sequence comprises sequencing TCRβ genes from a peripheral blood mononucleated cell sample from the subject.

17. The method of claim 16, wherein obtaining a TCRβ gene sequence further comprises isolating T cells from the sample.

18. The method of claim 17, wherein isolating T cells is by cell sorting and/or RNA expression.

19. The method of claim 18, wherein T cells are non-regulatory T cells.

20. The method of claim 1, wherein the cell is a peripheral blood mononucleated cell from a subject having cancer.

21. The method of claim 20, wherein the cancer is selected from the group consisting of melanoma, prostate cancer, non-small cell lung cancer, mesothelioma, bladder cancer and renal cancer.

22. The method of claim 1, wherein the ICI therapy is selected from the group consisting of ipilimumab, tremelimumab, atezolizumab, avelumab, durvalumab, nivolumab, pembrolizumab, ipilimumab plus nivolumab, and combinations thereof.


SEQUENCE LISTING (23 sequences)

SEQ ID NO: 1 - DNA, 27 bp, synthetic construct
  source 1..27: mol_type = other DNA
tgtgccagcaggggggaggttgacacc

SEQ ID NO: 2 - protein, 9 aa, synthetic construct
CASRGEVDT

SEQ ID NO: 3 - DNA, 25 bp, synthetic construct
  source 1..25: mol_type = other DNA
tgtgccaccgtatggagggaagtac

SEQ ID NO: 4 - DNA, 24 bp, synthetic construct
  source 1..24: mol_type = other DNA
tgtgccacctatggagggaagtac

SEQ ID NO: 5 - protein, 8 aa, synthetic construct
CATYGGKY

SEQ ID NO: 6 - DNA, 24 bp, synthetic construct
  source 1..24: mol_type = other DNA
tgtgccaccgatggagggaagtac

SEQ ID NO: 7 - protein, 8 aa, synthetic construct
CATDGGKY

SEQ ID NO: 8 - DNA, 24 bp, synthetic construct
  source 1..24: mol_type = other DNA
tgtgccaccgttggagggaagtac

SEQ ID NO: 9 - protein, 8 aa, synthetic construct
CATVGGKY

SEQ ID NO: 10 - DNA, 24 bp, synthetic construct
  source 1..24: mol_type = other DNA
tgtgccaccgtaggagggaagtac

SEQ ID NO: 11 - DNA, 24 bp, synthetic construct
  source 1..24: mol_type = other DNA
tgtgccaccgtatggagggagtac

SEQ ID NO: 12 - protein, 8 aa, synthetic construct
CATVWREY

SEQ ID NO: 13 - DNA, 27 bp, synthetic construct
  source 1..27: mol_type = other DNA
tgtgccagcagtgacgggacttagtac

SEQ ID NO: 14 - protein, 7 aa, synthetic construct
CASSDGT

SEQ ID NO: 15 - DNA, 27 bp, synthetic construct
  source 1..27: mol_type = other DNA
tgtgccagcagtgacgggactaagtac

SEQ ID NO: 16 - protein, 9 aa, synthetic construct
CASSDGTKY

SEQ ID NO: 17 - DNA, 27 bp, synthetic construct
  source 1..27: mol_type = other DNA
tgtgccagcagtgacgggactcagtac

SEQ ID NO: 18 - protein, 9 aa, synthetic construct
CASSDGTQY

SEQ ID NO: 19 - DNA, 27 bp, synthetic construct
  source 1..27: mol_type = other DNA
tgtgccagcagtgacgggactgagtac

SEQ ID NO: 20 - protein, 9 aa, synthetic construct
CASSDGTEY

SEQ ID NO: 21 - protein, 8 aa, synthetic construct
KDRSGTPY

SEQ ID NO: 22 - protein, 12 aa, synthetic construct
SHLWPTGATNEK

SEQ ID NO: 23 - protein, 9 aa, synthetic construct
SVGGTGANE